(19) United States
(12) Patent Application Publication
(10) Pub. No.: US 2016/0139149 A1
(43) Pub. Date: May 19, 2016

(54) CHI3L1 FOR THE DETECTION AND TREATMENT OF NONALCOHOLIC STEATOHEPATITIS

(71) Applicants: BROWN UNIVERSITY, Providence, RI (US); YALE UNIVERSITY, New Haven, CT (US); RHODE ISLAND HOSPITAL, Providence, RI (US)

(72) Inventors: Jack A. ELIAS, Providence, RI (US); Jack R. WANDS, East Greenwich, RI (US); Chun Geun LEE, Woodbridge, CT (US); Kittichai PROMRAT, Rumford, RI (US)

(73) Assignees: BROWN UNIVERSITY, Providence, RI (US); RHODE ISLAND HOSPITAL, Providence, RI (US); YALE UNIVERSITY, New Haven, CT (US)

(21) Appl. No.: 14/945,759

(22) Filed: Nov. 19, 2015

Related U.S. Application Data
(60) Provisional application No. 62/081,913, filed on Nov. 19, 2014.

(51) Int. Cl. G01N 33/68; C12Q 1/68; C12N 15/113

(52) U.S. Cl. CPC G01N 33/6893; C12N 15/113; C12N 15/1138; C12Q 1/6883; C12N 2310/531; C12N 2310/14; C12N 2320/30; C12Q 2600/118; C12Q 2600/158; G01N 2333/4703; G01N 2800/085; G01N 2800/52

(57) ABSTRACT

Disclosed herein are compositions and methods for detecting nonalcoholic steatohepatitis (NASH) measuring CHI3L1 levels. In some embodiments, the methods described herein can distinguish NASH from nonalcoholic fatty liver disease. CHI3L1 levels can also be used to monitor treatment progress in patients having NASH. Also disclosed herein are compositions and methods for treating NASH by targeting CHI3L1.

### CROSS REFERENCE PARAGRAPH

This application claims benefit under 35 U.S.C. §119(e) of U.S. Provisional Application No. 62/081,913 filed Nov. 19, 2014, the contents of which are incorporated herein by reference in its entirety.

### GOVERNMENT SUPPORT

This invention was made with government support under grant U01-HL-108638 awarded by the National Heart, Lung and Blood Institute (NHLBI) and National Institutes of Health (NIH). The government has certain rights in the invention.

### TECHNICAL FIELD

The present disclosure relates to the detection and treatment of nonalcoholic steatohepatitis.

### BACKGROUND

Nonalcoholic steatohepatitis or NASH is a common, often “silent” liver disease. It resembles alcoholic liver disease, but occurs in people who drink little or no alcohol. The major feature in NASH is fat in the liver, along with inflammation and damage. The severity of NASH can be assessed by the NASH activity index (NAI). NASH is the progressive form of the relatively benign non-alcoholic fatty liver disease (NAFLD). NASH can be severe and can lead to cirrhosis, in which the liver is permanently damaged and scarred and no longer able to work properly. According to the National Institutes of Health, NASH affects 2 to 5 percent of Americans.

The current gold standard to confirm NASH is a histological evaluation of liver biopsy, which is expensive, invasive, and can cause pain, hemorrhage, or even death. A simple blood test that would identify NASH and/or distinguish NASH from NAFLD (and thereby reduce the need for liver biopsy) would be highly desirable.

### SUMMARY

The technology described herein is based, in part, on the discovery that CHI3L1 serum levels are elevated in patients with NASH and that treatment that lowers the NAI in the liver also reduces CHI3L1 serum levels.

One aspect of the technology described herein relates to an assay comprising: (1) measuring, in a sample obtained from a subject, a level of CHI3L1; (2) comparing the level of CHI3L1 with a reference level; and (3) identifying the subject as (a) having nonalcoholic steatohepatitis and not nonalcoholic fatty liver disease if the level of CHI3L1 is above the reference level, or (b) not having nonalcoholic steatohepatitis if the level of CHI3L1 is at or below the reference level.

One aspect of the technology described herein relates to a method of detecting nonalcoholic steatohepatitis in a subject, the method comprising: (1) assaying, in a sample obtained from a subject, a level of CHI3L1; (2) comparing the level of CHI3L1 with a reference level; and (3) identifying the subject as (a) having nonalcoholic steatohepatitis and not nonalcoholic fatty liver disease if the level of CHI3L1 is above the reference level, or (b) not having nonalcoholic steatohepatitis if the level of CHI3L1 is at or below the reference level.

Another aspect of the technology described herein relates to a method of distinguishing nonalcoholic steatohepatitis from nonalcoholic fatty liver disease, the method comprising: (1) assaying, in a sample obtained from a subject, a level of CHI3L1; (2) comparing the level of CHI3L1 with a reference level; and (3) identifying the subject as (a) having nonalcoholic steatohepatitis and not nonalcoholic fatty liver disease if the level of CHI3L1 is above the reference level, or (b) not having nonalcoholic steatohepatitis if the level of CHI3L1 is at or below the reference level.

In some embodiments of any of the foregoing aspects, when the level of CHI3L1 is above the reference level, the method further comprises providing a treatment appropriate for treating nonalcoholic steatohepatitis. In some embodiments of any of the foregoing aspects, the treatment comprises administering a CHI3L1 inhibitor.

In some embodiments of any of the foregoing aspects, the level of CHI3L1 is a protein level. In some embodiments of any of the foregoing aspects, the level of CHI3L1 is measured by an immunoassay. In some embodiments of any of the foregoing aspects, the sample is contacted with an anti-CHI3L1 antibody. In some embodiments of any of the foregoing aspects, the anti-CHI3L1 antibody is detectably labeled or capable of generating a detectable signal. In some embodiments of any of the foregoing aspects, the antibody is fluorescently labeled.

In some embodiments of any of the foregoing aspects, the level of CHI3L1 is measured by measuring a nucleic acid encoding CHI3L1.

In some embodiments of any of the foregoing aspects, the reference level is an average CHI3L1 level in a population of healthy subjects. In some embodiments of any of the foregoing aspects, the reference level is two standard deviations above an average CHI3L1 level in a population of healthy subjects.

In some embodiments of any of the foregoing aspects, the sample is a blood, plasma, or serum sample.

One aspect of the technology described herein relates to a method of monitoring treatment progress in a subject having nonalcoholic steatohepatitis, the method comprising: (i) measuring, at a first time point, a first level of CHI3L1 in a first sample obtained from the subject; (ii) administering to the subject a therapeutic agent for treating nonalcoholic steatohepatitis; and (iii) measuring, at a second time point, a second level of CHI3L1 in a second sample obtained from the subject, wherein the second time point is later than the first time point and after said administering, and wherein if the second level is significantly lower than the first level, then the treatment is considered to be effective.

In some embodiments, the first sample and the second sample are blood, plasma, or serum samples. In some embodiments, the therapeutic agent is a CHI3L1 inhibitor.

Another aspect of the technology described herein relates to a method of treating nonalcoholic steatohepatitis in a subject, the method comprising administering a therapeutically-effective amount of a CHI3L1 inhibitor to the subject.

In some embodiments, the CHI3L1 inhibitor decreases the expression level of CHI3L1 protein.

In some embodiments, the CHI3L1 inhibitor decreases the activity of CHI3L1 protein.

In some embodiments of any of the foregoing aspects, the CHI3L1 inhibitor is selected from the group consisting of a small molecule, a nucleic acid, a nucleic acid analog or derivative, a peptide, a peptidomimetic, a protein, an antibody or an antigen-binding fragment thereof, a saccharide, a lipid, a glycosaminoglycan, an extract made from a biological material, and combinations thereof.

In some embodiments of any of the foregoing aspects, the subject is a mammal.

In some embodiments of any of the foregoing aspects, the subject is a human.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of refers to those elements required for a given embodiment. The term permits the presence of elements that do not materially affect the basic and novel or functional characteristic(s) of that embodiment of the invention.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

As used herein, “nonalcoholic steatohepatitis” or “NASH” refers to a progressive disease or disorder characterized by inflammation of the liver in combination with fatty liver. It resembles alcoholic liver disease, but occurs in people who drink little or no alcohol. NASH can lead to fibrosis, cirrhosis, end stage liver disease, hepatic failure, and hepatocellular carcinoma.

As used herein, “nonalcoholic fatty liver disease” or “NAFLD” refers to a benign and nonprogressive disease or disorder characterized in abnormal fat deposition (i.e. steatosis) in the liver not due to excessive alcohol use.

As used herein, “IL-13Rα2,” “Interleukin-13 receptor subunit alpha-2,” or “CD213A2” refers to a membrane bound protein that binds to IL-13 with very high affinity, and negatively regulates both IL-13 and IL-4. IL-13Rα2 competes with the IL-13 receptor comprising IL-13Rα1 and IL4R for binding of IL-13. Sequences for IL-13Rα2 expression products are known for a number of species, e.g., human IL-13Rα2 (NCBI Gene ID: 3598) mRNA (NCBI Ref Seq: NM_000640) and polypeptide (NCBI Ref Seq: NP_000631).

As used herein, “CHI3L1,” “chitinase-3-like protein 1,” or “YKL-40” refers to a ˜40 kDa glycoprotein secreted by at least macrophages, chondrocytes, neutrophils, synovial cells, and some cancer cells. CHI3L1 does not have chitinase activity, is a Th2 promoting cytokine, has been linked to the AKT anti-apoptotic signaling pathway and induces the migration of astrocytes. The sequences of CHI3L1 expression products are known for a number of species, e.g., human CHI3L1 (NCBI Gene ID No: 1116) mRNA (SEQ ID NO: 2; NCBI Ref Seq: NM 001276) and polypeptide (SEQ ID NO: 1; NCBI Ref Seq: NP 001267). The activity of CHI3L1 can be measured, e.g., by measuring the anti-apoptotic effects of CHI3L1, or by assaying promotion of Th2 cytokine production.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, CHI3L1, e.g. its ability to decrease the level and/or activity of CHI3L1 can be determined, e.g. by measuring the level of an expression product of CHI3L1 and/or the activity of CHI3L1. Methods for measuring the level of a given mRNA and/or polypeptide are known to one of skill in the art, e.g. RT-PCR can be used to determine the level of RNA, and Western blotting or immunoassay with an antibody (e.g. an anti-CHI3L1 antibody) can be used to determine the level of a polypeptide. The activity of, e.g. CHI3L1 can be determined using methods known in the art. In some embodiments, the inhibitor can be an inhibitory nucleic acid; an aptamer; an antibody reagent; an antibody; or a small molecule.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of NASH. A subject can be male or female.

A subject can be one who has been previously diagnosed with or identified as suffering from or having a condition in need of treatment (e.g. NASH) or one or more complications related to such a condition, and optionally, have already undergone treatment for NASH or the one or more complications related to NASH (e.g. cirrhosis). Alternatively, a subject can also be one who has not been previously diagnosed as having, e.g., NASH or one or more complications related to NASH. For example, a subject can be one who exhibits one or more risk factors for NASH or one or more complications related to NASH or a subject who does not exhibit risk factors.

A “subject in need” of treatment for a particular condition can be a subject having that condition, diagnosed as having that condition, or at elevated risk of developing that condition.

The term “sample”, “biological sample”, or “test sample” as used herein denotes a sample taken or isolated from a biological organism, e.g., a blood or plasma sample from a subject. Exemplary biological samples include, but are not limited to, a biofluid sample; serum; plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, the test sample can be a blood sample. In some embodiments, the test sample can be a plasma sample. In some embodiments, the test sample can be a serum sample.

The test sample can be obtained by removing a sample from a subject, but can also be accomplished by using previously sample (e.g. isolated at a prior time point and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site comprising the complementarity determining regions (CDRs) of an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab')2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized. In certain embodiments, specific binding is indicated by a dissociation constant on the order of ≦10⁻⁸ M, ≦10⁻⁹ M, ≦10⁻¹⁰ M or below.

As used herein, “expression level” refers to the number of mRNA molecules and/or polypeptide molecules encoded by a given gene that are present in a cell or sample. Expression levels can be increased or decreased relative to a reference level.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. NASH. The term “treating” includes reducing or alleviating at least one adverse effect or symptom of a condition, disease or disorder, e.g. NASH. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intrahepatic, intraperitoneal, transtracheal, subcutaneous, subcuticular, intraarticular, sub capsular, subarachnoid, intraspinal, intracerebro spinal, and intrasternal injection and infusion. The administration can be systemic or local.

The term “statistically significant” or “significantly” refers to statistical significance and generally means a two standard deviation (2SD) or greater difference.

As used herein, the term “significantly” should be interpreted as if modified by the term “statistically”.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows representative histology of liver on the mice fed with regular chow and MCD diet (NASH-inducing diet) for 24 days. Significant fat accumulation was noted in the mice fed with NASH chow compared to the mice fed with regular chow. Arrows indicates the fat globules.

FIG. 2 shows representative histology of liver on the mice fed with MCD diet (NASH-inducing diet) for 24 days along with the treatment of vehicle or lentiviral particles containing control shRNA, Chi3l1-specific shRNA or IL-13Rα2-specific ShRNA. The accumulated fat globules are easily recognized with trichrome staining as empty holes. The number and size of these fat globules were significantly decreased in the mice treated with Chi3l1- or IL-13Rα2-specific shRNA compared to the mice treated with vehicle or control lenti-shRNA.

FIG. 3 shows representative histology of liver on the mice fed with MCD diet (NASH-inducing diet) for 24 days in wild-type (WT) and Chi3l1 null mutant mice (Chi3l1 KO). H&E stains

FIG. 4 shows change in serum chitinase-3-like-1 protein level in NASH comparing lifestyle intervention vs control group.

FIG. 5 shows change in serum chitinase-3-like-1 protein level in NASH comparing individuals who successfully achieved weight loss goal (>7%) vs those who did not.

FIG. 6 shows change in serum chitinase-3-like-1 protein level in NASH comparing individuals who had improved histology after treatment vs those who did not. Histologic improvement is defined by a drop in Nonalcoholic Steatohepatitis Activity Score (NAS) of greater than or equal to 3 points from baseline or Post-Treatment NAS of lower than or equal to 2.

FIG. 7 is a diagram of an exemplary embodiment of a system for performing an assay for determining the level of CHI3L1 in a sample obtained from a subject.

FIG. 8 is a diagram of an exemplary embodiment of a comparison module as described herein.

FIG. 9 is a diagram of an exemplary embodiment of an operating system and applications for a computing system as described herein.

### DETAILED DESCRIPTION

The technology described herein is based, in part, on the discovery that CHI3L1 serum levels are elevated in patients with NASH and that treatment that lowers the NAI in the liver also reduces CHI3L1 serum levels. Accordingly, embodiments of the technology described herein are related to the use of CHI3L1 as a biomarker for the detection of NASH and as a target for the treatment of NASH. Additionally, some embodiments of the technology described herein are related to the use of CHI3L1 as a biomarker to distinguish NASH from NAFLD.

In one aspect, the technology described herein provides a method or assay, the method or assay comprising: (1) measuring, in a sample obtained from a subject, a level of CHI3L1; (2) comparing the level of CHI3L1 with a reference level; and (3) identifying the subject as (a) having nonalcoholic steatohepatitis and not nonalcoholic fatty liver disease if the level of CHI3L1 is above the reference level, or (b) not having nonalcoholic steatohepatitis if the level of CHI3L1 is at or below the reference level.

In some embodiments, the methods and assays described herein include (a) transforming the CHI3L1 into a detectable target; (b) measuring the amount of the target; and (c) comparing the amount of the target to an amount of a reference level, wherein if the amount of the detectable target is statistically significantly greater than the amount of the reference level, the subject is identified as having nonalcoholic steatohepatitis and not nonalcoholic fatty liver disease. In some embodiments, if the amount of the detectable target is not statistically significantly greater than the amount of the reference level, the subject is identified as not having nonalcoholic steatohepatitis.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Transformation, measurement, and/or detection of a target molecule, e.g. a CHI3L1 mRNA or polypeptide can comprise contacting a sample obtained from a subject with a reagent (e.g. a detection reagent) which is specific for the target, e.g., a CHI3L1-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure CHI3L1 gene expression products are well known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for CHI3L1 are commercially available and can be used for the purposes of the invention to measure protein expression levels, e.g. anti-CHI3L1 (Cat. No. ab86428; Abcam, Cambridge Mass.). Alternatively, since the amino acid sequences for CHI3L1 are known and publically available at NCBI website, one of skill in the art can raise their own antibodies against these polypeptides of interest for the purpose of the invention.

The amino acid sequences of the polypeptides described herein, e.g. CHI3L1 have been assigned NCBI accession numbers for different species such as human, mouse and rat. In particular, the NCBI accession numbers for the amino acid sequence of human CHI3L1 is included herein, e.g. SEQ ID NO: 1.

| human CHI3L1 polypeptide NCBI Ref Seq: NP_001267 | |
|---|---|
| SEQ ID NO: 1 | |
| 1 | mgvkasqtgf vvlvllqccs ayklvcyyts wsqyregdgs cfpdaldrfl cthiiysfan |
| 61 | isndhidtwe wndvtlygml ntlknrnpnl ktllsvggwn fgsqrfskia sntqsrrtfi |
| 121 | ksvppflrth gfdgldlawl ypgrrdkqhf ttlikemkae fikeaqpgkk qlllsaalsa |
| 181 | gkvtidssyd iakisqhldf isimtydfhg awrgttghhs plfrgqedas pdrfsntdya |
| 241 | vgymlrlgap asklvmgipt fgrsftlass etgvgapisg pgipgrftke agtlayyeic |
| 301 | dflrgatvhr ilgqqvpyat kgnqwvgydd qesvkskvqy lkdrqlagam vwaldlddfq |
| 361 | gsfcgqdlrf pltnaikdal aat |

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color or other readily detectable property, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., CHI3L1 as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′,5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent applications Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of, e.g., CHI3L1, can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein, e.g. CHI3L1. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNA protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some related technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein, e.g., CHI3L1, have been assigned NCBI accession numbers for different species such as human, mouse and rat. For example, the human CHI3L1 mRNA (e.g. SEQ ID NO: 2) is known. Accordingly, a skilled artisan can design an appropriate primer based on the known sequence for determining the mRNA level of the respective gene.

| human CHI3L1 mRNA NCBI Ref Seq: NM_001276 | |
|---|---|
| SEQ ID NO: 2 | |
| 1 | cacatagctc agttcccata aaagggctgg tttgccgcgt cggggagtgg agtgggacag |
| 61 | gtatataaag gaagtacagg gcctggggaa gaggccctgt ctaggtagct ggcaccagga |
| 121 | gccgtgggca agggaagagg ccacaccctg ccctgctctg ctgcagccag aatgggtgtg |
| 181 | aaggcgtctc aaacaggctt tgtggtcctg gtgctgctcc agtgctgctc tgcatacaaa |
| 241 | ctggtctgct actacaccag ctggtcccag taccgggaag gcgatgggag ctgcttccca |
| 301 | gatgcccttg accgcttcct ctgtacccac atcatctaca gctttgccaa tataagcaac |
| 361 | gatcacatcg acacctggga gtggaatgat gtgacgctct acggcatgct caacacactc |
| 421 | aagaacagga accccaacct gaagactctc ttgtctgtcg gaggatggaa ctttgggtct |
| 481 | caaagatttt ccaagatagc ctccaacacc cagagtcgcc ggactttcat caagtcagta |
| 541 | ccgccatttc tgcgcaccca tggctttgat gggctggacc ttgcctggct ctaccctgga |
| 601 | cggagagaca aacagcattt taccacccta atcaaggaaa tgaaggccga atttataaag |
| 661 | gaagcccagc cagggaaaaa gcagctcctg ctcagcgcag cactgtctgc ggggaaggtc |
| 721 | accattgaca gcagctatga cattgccaag atatcccaac acctggattt cattagcatc |
| 781 | atgacctacg attttcatgg agcctggcgt gggaccacag gccatcacag tcccctgttc |
| 841 | cgaggtcagg aggatgcaag tcctgacaga ttcagcaaca ctgactatgc tgtggggtac |
| 901 | atgttgaggc tgggggctcc tgccagtaag ctggtgatgg gcatccccac cttcgggagg |
| 961 | agcttcactc tggcttcttc tgagactggt gttggagccc caatctcagg accgggaatt |
| 1021 | ccaggccggt tcaccaagga ggcagggacc cttgcctact atgagatctg tgacttcctc |
| 1081 | cgcggagcca cagtccatag aatcctcggc cagcaggtcc cctatgccac caagggcaac |
| 1141 | cagtgggtag gatacgacga ccaggaaagc gtcaaaagca aggtgcagta cctgaaggac |
| 1201 | aggcagctgg cgggcgccat ggtatgggcc ctggacctgg atgacttcca gggctccttc |
| 1261 | tgcggccagg atctgcgctt ccctctcacc aatgccatca aggatgcact cgctgcaacg |
| 1321 | tagccctctg ttctgcacac agcacggggg ccaaggatgc cccgtccccc tctggctcca |
| 1381 | gctggccggg agcctgatca cctgccctgc tgagtcccag gctgagcctc agtctccctc |
| 1441 | ccttggggcc tatgcagagg tccacaacac acagatttga gctcagccct ggtgggcaga |
| 1501 | gaggtaggga tggggctgtg gggatagtga ggcatcgcaa tgtaagactc gggattagta |
| 1561 | cacacttgtt gattaatgga aatgtttaca gatccccaag cctggcaagg gaatttcttc |
| 1621 | aactccctgc cccccagccc tccttatcaa aggacaccat tttggcaagc tctatcacca |
| 1681 | aggagccaaa catcctacaa gacacagtga ccatactaat tataccccct gcaaagccca |
| 1741 | gcttgaaacc ttcacttagg aacgtaatcg tgtcccctat cctacttccc cttcctaatt |
| 1801 | ccacagctgc tcaataaagt acaagagctt aacagtgaaa aaaaaaaaaa aaaaaaaaaa |
| 1861 | aaaaaaa |

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylene diaminetetraacetic acid (EDTA).

A level which is greater than a reference level can be a level which is greater by at least about 10%, at least about 20%, at least about 50%, at least about 100%, at least about 200%, at least about 300%, at least about 500%, at least about 1000%, or greater than the reference level. In some embodiments, a level which is greater than a reference level can be a level which is statistically significantly greater than the reference level. In some embodiments, the reference level can be a level of CHI3L1 in a population of healthy subjects. This can be the “normal” level. In some embodiments, the reference level can be two standard deviations above an average CHI3L1 level in a population of healthy subjects. In some embodiments, the reference level can be an average level of CHI3L1 in a population of subjects who do not have or are not diagnosed as having, and/or do not exhibit signs or symptoms of NASH. In some embodiments, the reference level can be an average level of CHI3L1 in a population of subjects who have NAFLD. In some embodiments, the reference can also be a level of expression of CHI3L1 in a control sample, a pooled sample of control individuals or a numeric value or range of values based on the same. In some embodiments, the reference can be the level of CHI3L1 in a sample obtained from the same subject at an earlier point in time, e.g., the methods described herein can be used to monitor the treatment progress in a subject having NASH.

It should be noted that the reference level can be different, depending on factors such as the sample type from which the reference level is derived, gender, age, weight, and ethnicity. Thus, reference levels accounting for these and other variables can provide added accuracy for the assays, methods, and systems described herein.

In some embodiments, the method, assays, and systems described herein can further comprise a step of providing a treatment appropriate for treating NASH. Treatment for NASH includeS, but is not limited to, lifestyle changes such as weight loss and regular exercise; drugs that lower total cholesterol level such as statin.

In some embodiments, the methods, assays, and systems described herein can further comprise a step of obtaining a test sample from a subject. In some embodiments, the subject can be a human subject. In some embodiments, the subject can be a subject having liver disease. In some embodiments, the subject can be a subject in need of treatment for (e.g. having or diagnosed as having) NASH. In some embodiments, the subject can be a subject undergoing treatment for NASH.

Because the CHI3L1 level is significantly higher in subjects having NASH than those having NAFLD, the methods, assays, and systems described herein can be used to distinguish NASH from NAFLD.

CHI3L1 levels can also be used to monitor the treatment progress in a subject having NASH. In some embodiments, the method comprising: (i) measuring, at a first time point, a first level of CHI3L 1 in a first sample obtained from the subject; (ii) administering to the subject a therapeutic agent for treating NASH; and (iii) measuring, at a second time point, a second level of CHI3L1 in a second sample obtained from the subject, wherein the second time point is later than the first time point and after said administering, and wherein if the second level is significantly lower than the first level, then the treatment is considered to be effective.

In one aspect, described herein is a kit for performing any of the assays and/or methods described herein. In some embodiments, the kit can comprise a CHI3L1-specific reagent.

A kit is any manufacture (e.g., a package or container) comprising at least one reagent, e.g., an antibody reagent(s) or nucleic acid probe, for specifically detecting, e.g., a CHI3L1 expression product or fragment thereof, the manufacture being promoted, distributed, or sold as a unit for performing the methods or assays described herein. When the kits, and methods described herein are used for diagnosis and/or treatment of NASH, the reagents (e.g., detection probes) or systems can be selected such that a positive result is obtained in at least about 80%, at least about 90%, at least about 95%, at least about 99% or in 100% of subjects having NASH.

In some embodiments, described herein is a kit for the detection of a CHI3L1 expression product in a sample, the kit comprising at least a first CHI3L1-specific reagent as described herein which specifically binds the CHI3L1 expression product, on a solid support. The reagent can optionally comprise a detectable label. The kits described herein include reagents and/or components that permit assaying the level of an expression product in a sample obtained from a subject (e.g., a biological sample obtained from a subject). The kits described herein can optionally comprise additional components useful for performing the methods and assays described herein.

A kit can further comprise devices and/or reagents for concentrating an expression product (e.g, a polypeptide) in a sample, e.g. a serum sample. Thus, ultrafiltration devices permitting, e.g., protein concentration can also be included as a kit component.

Preferably, a diagnostic or prognostic kit for use with the methods and assays disclosed herein contains detection reagents for CHI3L1 expression products. Such detection reagents comprise in addition to CHI3L1-specific reagents, for example, buffer solutions, labels or washing liquids etc. Furthermore, the kit can comprise an amount of a known nucleic acid and/or polypeptide, which can be used for a calibration of the kit or as an internal control. A diagnostic kit for the detection of an expression product can also comprise accessory ingredients like secondary affinity ligands, e.g., secondary antibodies, detection dyes and any other suitable compound or liquid necessary for the performance of an expression product detection method known to the person skilled in the art. Such ingredients are known to the person skilled in the art and may vary depending on the detection method carried out. Additionally, the kit may comprise an instruction leaflet and/or may provide information as to the relevance of the obtained results.

In one aspect, the technology described herein is directed to systems (and computer readable media for causing computer systems) for obtaining data from at least one sample obtained from at least one subject, the system comprising 1) a measuring module configured to receive the at least one sample and perform at least one analysis on the at least one sample to determine the level and/or activity of CHI3L1 in the sample; 2) a storage device configured to store data output from the determination module; and 3) a display module for displaying a content based in part on the data output from the determination module, wherein the content comprises a signal indicative of the level and/or activity of CHI3L1.

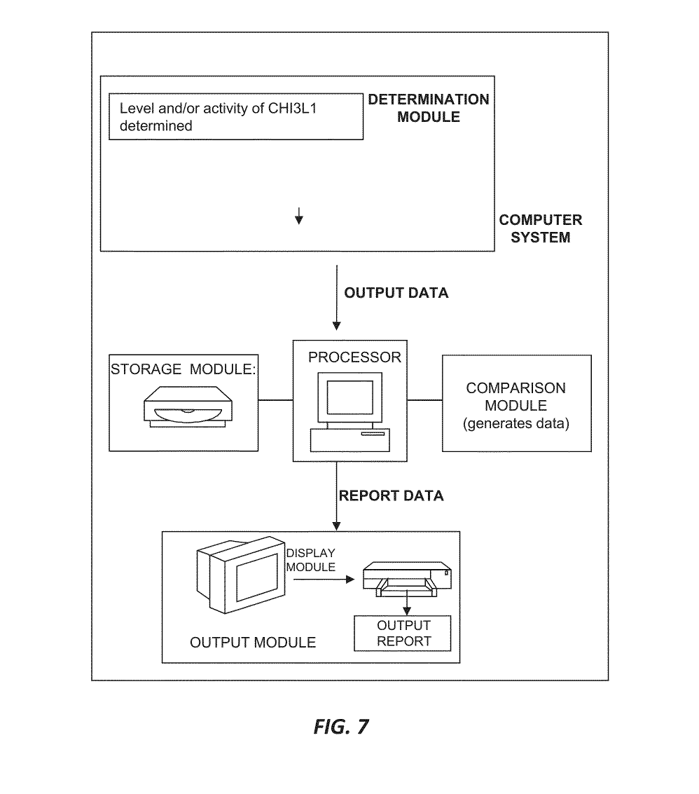

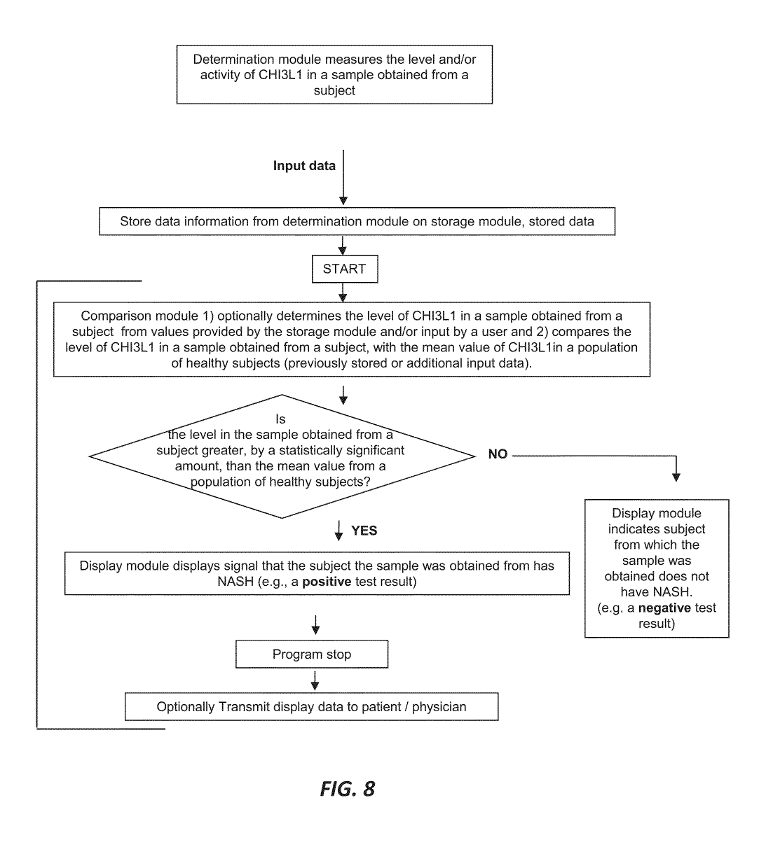

In one embodiment, provided herein is a system comprising: (a) at least one memory containing at least one computer program adapted to control the operation of the computer system to implement a method that includes a measuring module configured to measure the level of CHI3L1 in a test sample obtained from a subject; a storage module configured to store output data from the determination module; a comparison module adapted to compare the data stored on the storage module with a reference level, and to provide a retrieved content, and a display module for displaying whether the sample comprises a level of CHI3L1 which is significantly increased relative to the reference expression level and/or displaying the relative level of CHI3L and (b) at least one processor for executing the computer program (see FIG. 7).

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; a tablet; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of expression products etc in computer readable form.

The measuring module can comprise any system for detecting a signal elicited from an assay to determine the level and/or activity of CHI3L1 as described above herein. In some embodiments, such systems can include an instrument, e.g., AU2700 (Beckman Coulter Brea, Calif.) for quantitative measurement of polypeptides or e.g., a real time PCR machine, e.g. a LIGHTCYCLER™ (Roche). In some embodiments, the measuring module can measure the intensity of a detectable signal from an assay indicating the level of CHI3L1 polypeptide in the test sample. In some embodiments, the assay can be an immunoassay. In some embodiments, the measuring module can measure the intensity of a detectable signal from a RT-PCR assay indicating the level of CHI3L1 RNA transcript in the test sample.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the determination module. In additional embodiments, the storage module stores reference information such as levels of CHI3L1 in healthy subjects and/or a population of healthy subjects.

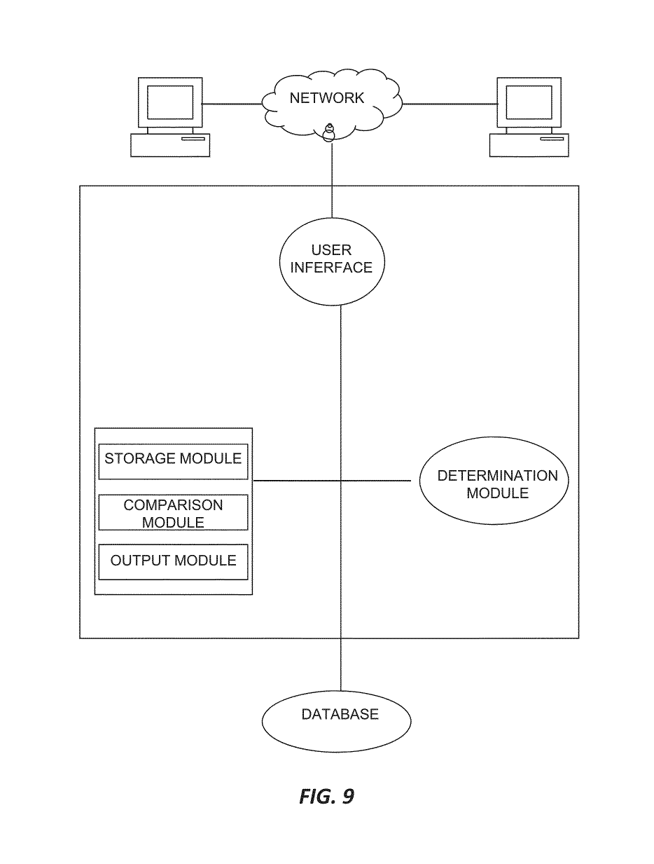

The “computing module” can use a variety of available software programs and formats for computing the level of CHI3L1. Such algorithms are well established in the art. A skilled artisan is readily able to determine the appropriate algorithms based on the size and quality of the sample and type of data. The data analysis tools and equations described herein can be implemented in the computing module of the invention. In one embodiment, the computing module further comprises a comparison module, which compares the level of CHI3L1 in a sample obtained from a subject as described herein with the mean value of CHI3L1 in a population of healthy subjects (FIG. 8). By way of an example, when the value of CHI3L1 in a sample obtained from a subject is measured, a comparison module can compare or match the output data with the mean value of CHI3L1 in a population of healthy subjects. In certain embodiments, the mean value of CHI3L1 in a population of healthy subjects can be pre-stored in the storage module. In various embodiments, the comparison module can be configured using existing commercially-available or freely-available software for comparison purpose, and may be optimized for particular data comparisons that are conducted.

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 9).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be the level of CHI3L1 in the sample obtained from a subject. In some embodiments, the content displayed on the display module can be the relative level of CHI3L1 in the sample obtained from a subject as compared to the mean level of CHI3L1 in a population of healthy subjects. In some embodiments, if the computing module determines that the level of CHI3L1 in the test sample obtained from a subject is greater by a statistically significant amount than the reference level, the display module displays a signal indicating that the levels in the sample obtained from a subject are greater than those of the reference level. In some embodiments, the signal indicates the subject is in need of treatment for NASH. In some embodiments, the signal indicates the degree to which the level of CHI3L1 in the sample obtained from a subject varies from the reference level. In some embodiments, the content displayed on the display module can indicate whether the subject has an increased likelihood of having or developing NASH. In some embodiments, the content displayed on the display module can be a numerical value indicating one of these risks or probabilities. In such embodiments, the probability can be expressed in percentages or a fraction. For example, higher percentage or a fraction closer to 1 indicates a higher likelihood of a subject having or developing NASH. In some embodiments, the content displayed on the display module can be single word or phrases to qualitatively indicate a risk or probability. For example, a word “unlikely” can be used to indicate a lower risk for having or developing NASH, while “likely” can be used to indicate a high risk for having or developing NASH.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for determining the level and/or activity of CHI3L1 in a sample obtained from a subject, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

In another aspect, the technology described herein provides a method of treating NASH in a subject, the method comprising administering a therapeutically-effective amount of a CHI3L1 inhibitor to the subject.

In another aspect, the technology described herein provides a method of treating NASH in a subject, the method comprising administering a therapeutically-effective amount of an IL-13Rα2 inhibitor to the subject.

A CHI3L1 or IL-13Rα2 inhibitor can have an IC50 of less than 50 μM, e.g., a CHI3L1 or IL-13Rα2 inhibitor can have an IC50 of from about 50 μM to about 5 nM, or less than 5 nM. For example, in some embodiments, a CHI3L1 inhibitor has an IC50 of from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

In some embodiments, the CHI3L1 inhibitor is a small molecule. In some embodiments, the IL-13Rα2 inhibitor is a small molecule. As used herein, the term “small molecule” refers to a natural or synthetic molecule having a molecular mass of less than about 5 kD, organic or inorganic compounds having a molecular mass of less than about 5 kD, less than about 2 kD, or less than about 1 kD.

In some embodiments, the CHI3L1 inhibitor can be an anti-CHI3L1 antibody molecule or an antigen-binding fragment thereof. In some embodiments, the IL-13Rα2 inhibitor can be an anti-IL-13Rα2 antibody molecule or an antigen-binding fragment thereof. Suitable antibodies include, but are not limited to, polyclonal, monoclonal, chimeric, humanized, recombinant, single chain, F_{ab}, F_{ab′}, F_{sc}, Rᵥ, and F_{(ab′)2} fragments. In some embodiments, neutralizing antibodies can be used as inhibitors of CHI3L1 or IL-13Rα2. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies. In general, an antibody molecule obtained from humans can be classified in one of the immunoglobulin classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies provide high binding avidity and unique specificity to a wide range of target antigens and haptens. Monoclonal antibodies useful in the practice of the methods disclosed herein include whole antibody and fragments thereof and are generated in accordance with conventional techniques, such as hybridoma synthesis, recombinant DNA techniques and protein synthesis.

The CHI3L1 and/or IL-13Rα2 polypeptide, or a portion or fragment thereof, can serve as an antigen, and additionally can be used as an immunogen to generate antibodies that immunospecifically bind the antigen, using standard techniques for polyclonal and monoclonal antibody preparation. Preferably, the antigenic peptide comprises at least 10 amino acid residues, or at least 15 amino acid residues, or at least 20 amino acid residues, or at least 30 amino acid residues.

Useful monoclonal antibodies and fragments can be derived from any species (including humans) or can be formed as chimeric proteins which employ sequences from more than one species. Human monoclonal antibodies or “humanized” murine antibody can also be used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction in the possibility of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is one option for minimizing the size of an antibody agent. ScFvs provide additional options for preparing and screening a large number of different antibody fragments to identify those that specifically bind. Techniques have been developed to display scFv molecules on the surface of filamentous phage that contain the gene for the scFv. scFv molecules with a broad range orantigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

In some embodiments, the CHI3L1 inhibitor is a nucleic acid or a nucleic acid analog or derivative thereof, also referred to as a nucleic acid agent herein. In some embodiments, the IL-13Rα2 inhibitor is a nucleic acid or a nucleic acid analog or derivative thereof. As will be appreciated by those skilled in the art, the depiction of a single strand also defines the sequence of the complementary strand. Thus, a nucleic acid also encompasses the complementary strand of a depicted single strand.

Without limitation, the nucleic acid agent can be single-stranded or double-stranded. A single-stranded nucleic acid agent can have double-stranded regions, e.g., where there is internal self-complementarity, and a double-stranded nucleic acid agent can have single-stranded regions. The nucleic acid can be of any desired length. In particular embodiments, nucleic acid can range from about 10 to 100 nucleotides in length. In various related embodiments, nucleic acid agents, single-stranded, double-stranded, and triple-stranded, can range in length from about 10 to about 50 nucleotides, from about 20 to about 50 nucleotides, from about 15 to about 30 nucleotides, from about 20 to about 30 nucleotides in length. In some embodiments, a nucleic acid agent is from about 9 to about 39 nucleotides in length. In some other embodiments, a nucleic acid agent is at least 30 nucleotides in length.

The nucleic acid agent can comprise modified nucleosides as known in the art. Modifications can alter, for example, the stability, solubility, or interaction of the nucleic acid agent with cellular or extracellular components that modify activity. In certain instances, it can be desirable to modify one or both strands of a double-stranded nucleic acid agent. In some cases, the two strands will include different modifications. In other instances, multiple different modifications can be included on each of the strands. The various modifications on a given strand can differ from each other, and can also differ from the various modifications on other strands. For example, one strand can have a modification, and a different strand can have a different modification. In other cases, one strand can have two or more different modifications, and the another strand can include a modification that differs from the at least two modifications on the first strand.

Single-stranded and double-stranded nucleic acid agents that are effective in inducing RNA interference are referred to as siRNA, RNAi agents, iRNA agents, or RNAi inhibitors herein. As used herein, the term “iRNA agent” refers to a nucleic acid agent which can mediate the targeted cleavage of an RNA transcript via an RNA-induced silencing complex (RISC) pathway.

In some embodiments, the CHI3L1 inhibitor is an antisense oligonucleotide. In some embodiments, the IL-13Rα2 inhibitor is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

A small hairpin RNA or short hairpin RNA (shRNA) is a sequence of RNA that makes a tight hairpin turn that can be used to silence target gene expression via RNA interference (RNAi). shRNAs that can be used to inhibit CHI3L1 or IL-13Rα2 are commercially available through venders such as Sigma Aldrich.

In general, any method of delivering a nucleic acid molecule can be adapted for use with the nucleic acid agents described herein.

Methods of delivering RNA interference agents, e.g., an siRNA, or vectors containing an RNA interference agent, to the target cells, for uptake include injection of a composition containing the RNA interference agent, e.g., an siRNA, or directly contacting the cell with a composition comprising an RNA interference agent, e.g., an siRNA. In another embodiment, RNA interference agent, e.g., an siRNA may be injected directly into any blood vessel, such as vein, artery, venule or arteriole, via, e.g., hydrodynamic injection or catheterization. Administration may be by a single injection or by two or more injections. The RNA interference agent is delivered in a pharmaceutically acceptable carrier. One or more RNA interference agents may be used simultaneously. In one embodiment, specific cells are targeted with RNA interference, limiting potential side effects. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. It is noted that RNA interference molecules tend to be taken up by the liver preferentially, rendering the treatment of NASH by targeting CHI3L1 more attractive via RNAi-based approaches than a disease or disorder that does not involve liver tissue. See, e.g., US20140194493 and US20130158097 for methods and compositions for delivering RNA interference agents to the liver. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, inhibit annealing of single strands, stabilize single strands, or otherwise facilitate delivery to the target cell and increase inhibition of the target gene, e.g., CHI3L1. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

In some embodiments, the CHI3L1 or IL-13Rα2 inhibitor can also be a peptide, a peptidomimetic, a protein, a saccharide, a lipid, a glycosaminoglycan, an extract made from a biological material, or combinations thereof.

In some embodiments, the CHI3L1 inhibitor prevents CHI3L1 protein from binding IL-13Rα2. In some embodiments, the CHI3L1 inhibitor reduces the probability of CHI3L1 protein binding IL-13Rα2.

In some embodiments, the inhibitors described herein can be formulated as a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl)Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), which are incorporated by reference herein in their entireties.

The CHI3L1 and/or IL-13Rα2 inhibitor can be administered in a pharmaceutical composition. In some embodiments, the pharmaceutical composition comprises a pharmaceutically-acceptable carrier and/or diluent. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The pharmaceutical compositions can be specially formulated for administration in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally the compounds described herein can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

The pharmaceutical compositions may be administered in any dose or dosing regimen. With respect to the therapeutic methods of the invention, it is not intended that the administration be limited to a particular mode of administration, dosage, or frequency of dosing.

The compounds of the present invention can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

In one embodiment, it may be desirable to administer the pharmaceutical compositions locally to the area in need of treatment; this may be achieved, for example, and not by way of limitation, by local infusion during surgery, topical application, e.g., by injection, by means of a catheter (e.g., a cardiac catheter, renal catheter, intrahepatic catheter, etc.), or by means of an implant, the implant being of a porous, non-porous, or gelatinous material, including membranes, such as sialastic membranes, fibers, or commercial skin substitutes. In some embodiments, for certain soft-tissue calcification site accessible by injection, an injection into the calcification site or its vicinity can be desirable.

In some embodiments, the pharmaceutical composition can be administered to a subject orally (e.g., in capsules, suspensions or tablets) or by parenteral administration. Conventional methods for oral administration include any one of the following; tablets, suspensions, solutions, emulsions, capsules, powders, syrups and the like are usable. Parenteral administration can include, for example, intramuscular, intravenous, intraarticular, intraarterial, intrathecal, subcutaneous, or intraperitoneal administration. The pharmaceutical composition can also be administered orally, transdermally, topically, by inhalation (e.g., intrabronchial, intranasal, oral inhalation or intranasal drops) or rectally.

When administering the pharmaceutical composition parenterally, it will generally be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and vegetable oils. The term “Dosage unit” form as used herein refers to physically discrete units suited as unitary dosages for the subjects to be treated; each unit containing a predetermined quantity of active compound calculated to produce the desired therapeutic effect in association with the required pharmaceutical carrier.

An effective amount, e.g., a therapeutically effective dose of the compound disclosed herein may be administered to the patient in a single dose or in multiple doses. When multiple doses are administered, the doses may be separated from one another by, for example, one hour, three hours, six hours, eight hours, one day, two days, one week, two weeks, or one month. For example, a composition comprising the compound disclosed herein can be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

The term “effective amount” as used herein refers to the amount of a therapy needed to alleviate at least one or more symptoms of the disease or disorder (e.g., NASH), and relates to a sufficient amount of pharmaceutical composition to provide the desired effect. The term “therapeutically effective amount” therefore refers to an amount of a therapy that is sufficient to cause a particular effect when administered to a typical subject. An effective amount as used herein, in various contexts, would also include an amount sufficient to delay the development of a symptom of the disease, alter the course of a symptom of the disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

In some embodiments, an effective amount of a CHI3L1 inhibitor can be an amount which causes the level of CHI3L1 expression to decrease or, at least, to increase at a lower rate than it would be expected to increase in a subject not receiving the CHI3L1 inhibitor. In some embodiments, an effective amount can be an amount that decreases the amount of CHI3L1 polypeptide present in the subject by a statistically significant amount. In some embodiments, an effective amount of a CHI3L1 inhibitor can be an amount which reduces the activity of CHI3L1 polypeptide. In some embodiments, an effective amount of a CHI3L1 inhibitor can be an amount which lowers the NAI by at least 5%, at least 10%, at least 20%, at least 30%, at least 40%, at least 50%, at least 60%, at least 70%, at least 80%, or at least 90%.

In some embodiments, an effective amount of an IL-13Rα2 inhibitor can be an amount which causes the level of IL-13Rα2 expression to decrease or, at least, to increase at a lower rate than it would be expected to increase in a subject not receiving the IL-13Rα2 inhibitor. In some embodiments, an effective amount can be an amount that decreases the amount of IL-13Rα2 polypeptide present in the subject by a statistically significant amount. In some embodiments, an effective amount of an IL-13Rα2 inhibitor can be an amount which reduces the activity of IL-13Rα2 polypeptide. In some embodiments, an effective amount of an IL-13Rα2 inhibitor can be an amount which lowers the NAI by at least 5%, at least 10%, at least 20%, at least 30%, at least 40%, at least 50%, at least 60%, at least 70%, at least 80%, or at least 90%.

A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the composition being administered, and the condition of the patient, the particular condition of soft-tissue calcification to be treated, as well as the body weight or body surface area. The size of the dose is also determined by the existence, nature, and extent of any adverse side-effects that accompany the administration of a particular formulation, or the like in a particular subject. Therapeutic compositions are optionally tested in one or more appropriate in vitro and/or in vivo animal models of disease, and known to persons of ordinary skill in the art, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of the pharmaceutical composition at various concentrations, e.g., as applied to the mass and overall health of the patient.

The dosage can be determined by one of skill in the art and can also be adjusted by the individual physician in the event of any complication. Typically, the dosage of a composition comprising the compound disclosed herein can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, or from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, or from 4.5 g/kg body weight to 5 g/kg body weight. Effective doses may be extrapolated from dose-response curves derived from in vitro or animal model test bioassays or systems. The dosage should not be so large as to cause unacceptable adverse side effects.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Although any known methods, devices, and materials may be used in the practice or testing of the invention, the methods, devices, and materials in this regard are described herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Example

### Inhibition of Diet Induced Non-Alcoholic Steatohepatitis (NASH) by Specific Silencing of Chi3l1 or IL-13Rα2 and Null Mutations of Chi3l1

Chi3l1 and its receptor IL-13Rα2 play significant roles in the pathogenesis of NASH. An established animal model of NASH and lenti-shRNA that specifically silence Chi3l1 or IL-13Rα2 were used. The diet induced NASH responses in wild type and Chi3l1 null mice were also characterized.

### Materials and Methods

NASH animal model: Six- to eight-week-old C57BL/6 male mice were used for the experiment. To induce NASH, the mice were fed with a methionine-choline-deficient (MCD) diet for 24 days according to the protocols reported previously (Henao-Mejia, J., et al., Nature 2012, 482:179-185).

Selection of Chi3l1- and IL-13Rα2-specific lenti ShRNA and control lenti-shRNA: Predesigned Mission shRNA transduction particles (Sigma-Aldrich, St Louis, Mo.) were used for silencing of specific target genes. The catalog No. for CHI3L1 MISSION shRNA Lentiviral Transduction Particles is TRCN0000067806. The catalog No. for Control shRNA is SHC016V. Highly efficient and highly specific silencing lenti-shRNAs against Chi3l1 or IL-13Rα2 were chosen based on in vitro evaluations using macrophages (AMJ-C11, ATCC CRL-2456) and epithelial cell lines (MLE12 cells, ATCC CRL-2110) after stimulation with rIL-13 (20 ng/ml). Among multiple candidates in the library, lenti-ShRNA TRCN0000111238 and TRCN000067806 were selected for Chi3l1 and IL-13Rα2, respectively, based on their ability to cause >90% and specific silencing as assessed by real-time qRT-PCR. Non-target shRNA control transduction particles (Cat #: SHC016V, sigma) were used as a control lenti-shRNA.

In vivo administration of Chi3l1 and IL-13Rα2 lenti-ShRNA: Mice were given 107 pfu/mouse lenti viral particles containing specific shRNA against Chi3l1- or IL-13Rα2 or controls via tail vein injection on days −1 and day 7 of MCD diet administration.

NASH in Chi3l1 null mice. Six- to eight-week-old C57BL/6 wild type and Chi3l1 null mice were fed with a methionine-choline-deficient (MCD) diet for 24-30 days as described above.

### Results

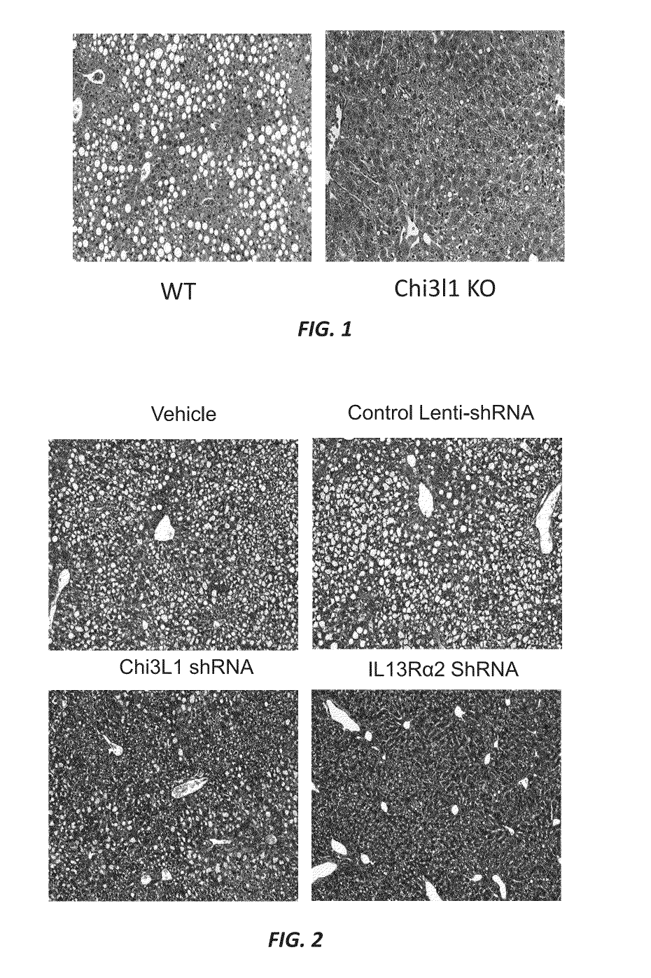

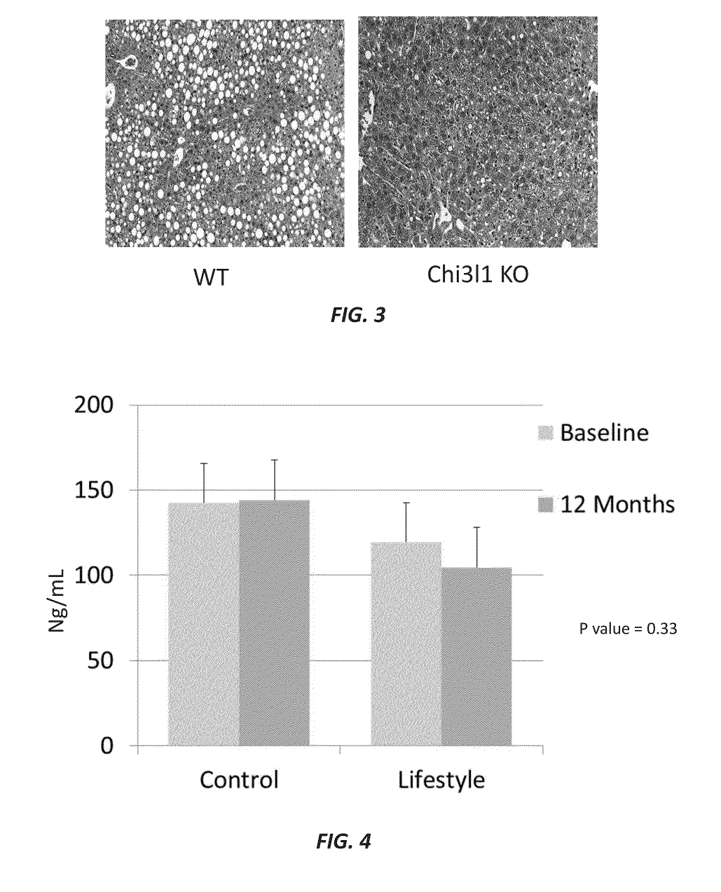

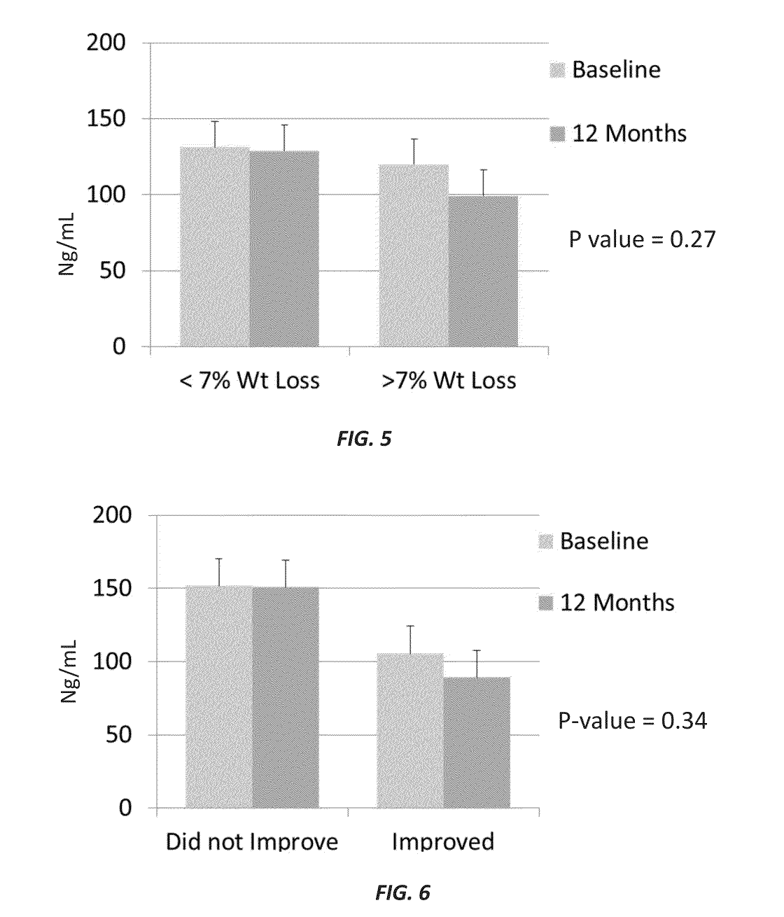

The mice fed a MCD diet showed significant increases in fat and inflammatory cell accumulation in the liver when compared to mice on regular chow (FIG. 1). Treatment with Chi3l1- or IL-13Rα2-specific lenti-shRNA diminished these MCD diet-induced responses (FIG. 2). Similarly, null mice with null mutations of Chi3l1 had decreased diet induced NASH responses (FIG. 3).

Chi3l1 and its receptor IL-13Rα2 are useful therapeutic targets for the control of NASH.

Data related to the Chi3l1 level in NASH after lifestyle intervention are presented in Tables 1-3.

TABLE 1
Correlation between Chitinase-3-like-1 Protein Levels (ng/mL) and
various variable from baseline and end of treatment (12 months).

| | | | | |
|---|---|---|---|---|
| | Baseline | | 12 Months | |
| Variable | Pearson r | P-value | Pearson r | P-value |

Physical Measurements

| | | | | |
|---|---|---|---|---|
| Weight (kg) | .05 | .809 | .17 | .357 |
| Waist Circumference | .09 | .621 | .16 | .401 |
| (cm) | | | | |
| BMI (kg/m2) | .15 | .426 | .21 | .272 |

Laboratory

| | | | | |
|---|---|---|---|---|
| ALT (U/L) | −.09 | .638 | .06 | .753 |
| AST (U/L) | .12 | .520 | .09 | .651 |
| Glucose (mg/dL) | .44 | .013** | .31 | .100 |
| Insulin | −.05 | .786 | −.06 | .769 |
| HOMA | .02 | .908 | −.05 | .811 |
| HbA1c | .27 | .138 | .32 | .086* |

Liver Biopsy

| | | | | |
|---|---|---|---|---|
| NAS score | .36 | .046** | .46 | .014** |
| Steatosis | .25 | .170 | .36 | .063* |
| Inflammation | .04 | .823 | .23 | .248 |
| Ballooning | .34 | .063* | .50 | .007** |
| Fibrosis | .44 | .013** | .25 | .194 |

In Table 1,
*Trend toward significance;
**Correlation is significant at the 0.05 level (2-tailed).

TABLE 2
Change in Chitinase-3-like-1 Protein Levels (ng/mL) from
Baseline to 12 months After Lifestyle Intervention.

| | N | Mean (SD) | p-value |
|---|---|---|---|
| Treatment | | | |
| Lifestyle | 20 | −14.8 (33.0) | |
| Control | 10 | 1.7 (59.2) | |
| Total | 30 | −9.3 (43.2) | .33 |
| 7% Weight Loss | | | |
| Yes | 11 | −20.8 (36.3) | |
| No | 19 | −2.6 (46.3) | |
| Total | 30 | −9.3 (43.2) | .27 |
| NAS Improvement at least 3 or | | | |
| NAS of 2 or less at post- | | | |
| treatment | | | |
| (ITT: ‘No’ assumed for | | | |
| missing) | | | |
| Yes | 16 | −16.4 (31.1) | |
| No | 14 | −1.2 (53.9) | |
| Total | 30 | −9.3 (43.2) | .34 |
| NAS Improvement at least 3 or | | | |
| NAS of 2 or less at post- | | | |
| treatment | | | |
| (Completers) | | | |
| Yes | 16 | −16.4 (31.1) | |
| No | 12 | −6.0 (53.9) | |
| Total | 28 | −11.9 (42.4) | .53 |

TABLE 3
Correlation between Change in Chitinase-3-like-1 Protein Levels
(ng/mL) from Baseline to 12 months and NAS change, % Weight
Change, and BMI change over the same period.

| Variable | N | Pearson r | P-value |
|---|---|---|---|
| NAS score | 28 | .37 | .053* |
| % Weight Change | 30 | .14 | .458 |
| BMI change | 30 | .16 | .408 |

1. An assay comprising:
(i) measuring, in a sample obtained from a subject, a level of CHI3L1;
(ii) comparing the level of CHI3L1 with a reference level; and
(iii) identifying the subject as (a) having nonalcoholic steatohepatitis and not nonalcoholic fatty liver disease if the level of CHI3L1 is above the reference level; or (b) not having nonalcoholic steatohepatitis if the level of CHI3L1 is at or below the reference level.

2. The assay of claim 1, wherein when the level of CHI3L1 is above the reference level, the assay further comprises providing a treatment appropriate for treating nonalcoholic steatohepatitis.

3. The assay of claim 2, wherein the treatment comprises administering a CHI3L1 inhibitor.

4. The assay of claim 1, wherein the level of CHI3L1 is a protein level.

5. The assay of claim 4, wherein the level of CHI3L1 is measured by an immunoassay.

6. The assay of claim 1, wherein the level of CHI3L1 is measured by measuring a nucleic acid encoding CHI3L1.

7. The assay of claim 1, wherein the reference level is an average CHI3L1 level in a population of healthy subjects.

8. A method of distinguishing nonalcoholic steatohepatitis from nonalcoholic fatty liver disease, the method comprising:
(i) assaying, in a sample obtained from a subject, a level of CHI3L1;
(ii) comparing the level of CHI3L1 with a reference level; and
(iii) identifying the subject as (a) having nonalcoholic steatohepatitis and not nonalcoholic fatty liver disease if the level of CHI3L1 is above the reference level; or (b) not having nonalcoholic steatohepatitis if the level of CHI3L1 is at or below the reference level.

9. The method of claim 8, wherein when the level of CHI3L1 is above the reference level, the method further comprises providing a treatment appropriate for treating nonalcoholic steatohepatitis.

10. The method of claim 9, wherein the treatment comprises administering a CHI3L1 inhibitor.

11. The method of claim 8, wherein the level of CHI3L1 is a protein level.

12. The method of claim 11, wherein the level of CHI3L1 is measured by an immunoassay.

13. The method of claim 8, wherein the level of CHI3L1 is measured by measuring a nucleic acid encoding CHI3L1.

14. The method of claim 8, wherein the reference level is an average CHI3L1 level in a population of healthy subjects.

15. A method of monitoring treatment progress in a subject having nonalcoholic steatohepatitis, the method comprising:
(i) measuring, at a first time point, a first level of CHI3L1 in a first sample obtained from the subject;
(ii) administering to the subject a therapeutic agent for treating nonalcoholic steatohepatitis; and
(iii) measuring, at a second time point, a second level of CHI3L1 in a second sample obtained from the subject, wherein the second time point is later than the first time point and after said administering, and wherein if the second level is significantly lower than the first level, then the treatment is considered to be effective.

16. The method of claim 15, wherein the therapeutic agent is a CHI3L1 inhibitor.

17. A method of treating nonalcoholic steatohepatitis in a subject, the method comprising administering a therapeutically-effective amount of a CHI3L1 inhibitor to the subject.

18. The method of claim 17, wherein the CHI3L1 inhibitor decreases the expression level of CHI3L1 protein.

19. The method of claim 17, wherein the CHI3L1 inhibitor decreases the activity of CHI3L1 protein.


SEQUENCE LISTING (2 sequences)

SEQ ID NO: 1 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 2 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa